(19) United States
(12) Patent Application Publication
(10) Pub. No.: US 2007/0117982 A1
(43) Pub. Date: May 24, 2007

(54) Method for producing 1,2-unsaturated azasteroids

(75) Inventors: Muhamed Jasic, Vienna (AT); Herbert Kollmann, Linz (AT); Bodo Lachmann, Frankfurt (DE); Christian Noe, Vienna (AT); Karin Zobl, Vienna (AT)

Correspondence Address: LERNER GREENBERG STEMER LLP, P O BOX 2480, HOLLYWOOD, FL, 33022-2480, US

(73) Assignee: Pharmacon Forschung und Beratung GmbH

(21) Appl. No.: 11/480,708

(22) Filed: Jul. 3, 2006

(30) Foreign Application Priority Data
Jan. 2, 2004 (AT) 4/2004

(51) Int. Cl. C07D 221/18

(52) U.S. Cl. 546/77

(57) ABSTRACT

The invention relates to a method for producing 1,2-unsaturated azasteroids of general formula (I), from a structurally related, saturated azasteroid of general formula (II).

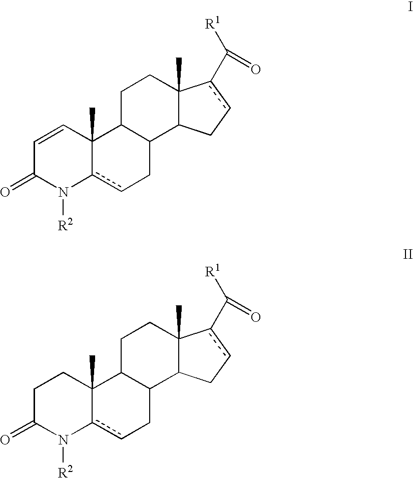

The present invention relates to the production of 2-unsaturated azasteroids and the use thereof for producing medicaments.

Azasteroids are compounds with a high potential for pharmacological effect. Various representatives of this class of compound have been approved as medicaments for a relatively long period (finasteride) or recently (turosteride). A series of methods of producing the pharmaceutically employed azasteroids, primarily for the active ingredient finasteride, has become known. Their synthesis starts from commercially available steroid intermediates (e.g. pregnenolone acetate, androstenolone acetate), which for their part are usually obtained from vegetable sources.

Of particular importance is the insertion of a double bond in ring A of the steroid basic structure between carbons 1 and 2 since, by doing so, for example in the case of the medicament finasteride, it was possible to achieve an increase in effectiveness and a reduction in the single dose. A series of direct and indirect (indirectly via electrophilic addition/substitution with subsequent elimination) oxidation methods have become known and are the subject matter of numerous patent specifications. However, only small yields are often quoted for these methods, or toxic reagents, such as phenylselenic anhydride or diphenyl diselenide, are used.

As regards azasteroids with antiandrogenic properties, the insertion of the 1,2 double bond into the compound 4-methyldihydrofinasteride (general formula II: R¹=NR^{2′}R^{3′}, where R^{2′} is H and R^{3′} is tert-butyl, R²=methyl, in which - - - marks single bonds and the A and B ring of the steroid are trans linked) through indirect oxidation has become known, where firstly the starting material is deprotonated with the base lithium diisopropylamide, than a thiophenyl group is inserted by means of diphenyl disulfide. Following purification by column chromatography, the thioether group is oxidized by means of sodium metaperiodate and the resulting intermediate is heated for the elimination in toluene. The end product was obtained following purification by column chromatography. The insertion of the 1,2 double bond into an azasteroid by the disulfide method is also described by reference to the synthesis of the 3-O-lactim ether of dihydrofinasteride. This 3-O-methyl group can be regarded as protective group (for avoiding the use of a further excess of lithium diisopropylamide for deprotonation of the proton on the nitrogen in position 4). After dehydrogenation, finasteride can be obtained through cleavage of the lactime ether. The method proceeds in a complicated manner and in poor yields. Considering the difficult handling and the price of organolithiums, the poor yields and the complexity of work-up and purification by column chromatography, it is no wonder that the disulfide method has not subsequently been pursued further and there has resulted instead the mentioned diversity of synthesis patents.

In the course of our own investigations, the inefficiency of using lithium diisopropylamide/diphenyl disulfide and the published synthesis procedures for inserting the 1,2 double bond into azasteroids was confirmed.

The basis of the present invention lies in the use of alkali metal alkoxides in inert solvents, which has proven not only to be optimal for the targeted deprotonation of azasteroids in the alpha position relative to the carbonyl group, but also, in contrast to the previously published methods, allows the 1,2-dehydrogenation to proceed in very good to excellent yields. The use of alkoxides dispenses with the use of difficult-to-handle and expensive organolithiums. The high yield during the production of the intermediate also permits its isolation and pure production and makes that of the end product significantly easier, without complex separation procedures. To achieve the required pharmaceutical grade, impurities must be able to be separated off from medicaments in the one tenth percent range. Since the dihydroazasterols themselves are a critical impurity of the azasterols, the high conversion rates during the reaction possible through the invention are of particular importance.

The present invention thus provides a method of producing 1,2-unsaturated azasteroids of the general formula I, 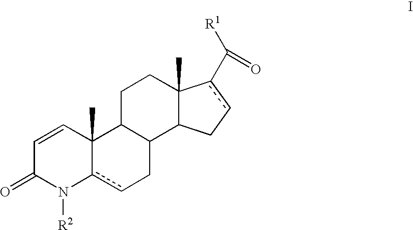
in which
R¹ is NR^{2′}R^{3′} (where R^{2′} and R^{3′}, which may be identical or different, may be H, branched or unbranched, saturated or unsaturated, substituted or unsubstituted lower alkyl having 1 to 7 carbon atoms, preferably tert-butyl, isopropyl, ethynyl, arylalkyl, benzyl, and (unsubstituted or substituted by branched or unbranched lower alkyl having 1 to 7 carbon atoms amino)carbonyl, preferably (isopropylamino)carbonyl), OR^{4′} (where R^{4′} is H, branched or unbranched, substituted or unsubstituted lower alkyl having 1 to 7 carbon atoms, preferably methyl), or nitrile,
R² is H
   and the bonds marked by - - - are single and double bonds in any combination,
   through efficient dehydrogenation of 1,2-saturated azasteroids of the general formula II. The method according to the invention comprises the following steps:
(a) reaction of a 1,2-saturated azasteroid of the general formula II, 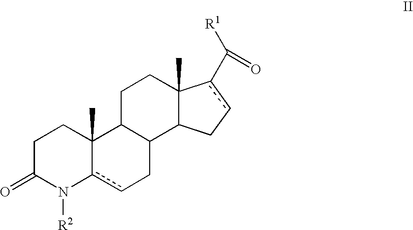
   in which
      R¹ is NR^{2′}R^{3′} (where R^{2′} and R^{3′}, which may be identical or different, may be H, branched or unbranched, saturated or unsaturated, substituted or unsubstituted lower alkyl having 1 to 7 carbon atoms, preferably tert-butyl, isopropyl, ethynyl, arylalkyl, benzyl, and (unsubstituted or substituted by branched or unbranched lower alkyl having 1 to 7 carbon atoms amino)carbonyl, preferably (isopropylamino)carbonyl), OR^{4′} (where R^{4′} is H, branched or unbranched, substituted or unsubstituted lower alkyl having 1 to 7 carbon atoms, preferably methyl), or nitrile, halogen or pyridiniummethyl,
   R² is H
   and the bonds marked by - - - are single and double bonds in any combination,
   as starting material with an alkali metal salt of a lower branched or unbranched alcohol in an inert solvent in the presence of an aryl disulfide of the general formula III,
      Ar—S—S—Ar III
   in which Ar is an unsubstituted or substituted aromatic ring,
(b) separating off the resulting thioether intermediate from remains of the starting material of the general formula II and from excess reagent of the formula III or from aromatic thiol,
(c) oxidation of the sulfur of the thioether intermediate,
(d) heating the resulting product in an inert solvent,
(e) isolation of the end product from the reaction mixture and
(f) if necessary, recrystallization of the end product obtained in this way.

According to one feature of the invention, the alkali metal salt of a lower branched or unbranched alcohol used in step (a) may be a sodium or potassium alkoxide. Preferred alkali metal alkoxides are sodium methoxide, sodium ethoxide and potassium tert-butoxide. One of the advantages of this invention is that the alkoxides to be used are not expensive and are easier to handle.

According to one feature of the invention, the inert solvent used in step (a) is a non-pure alcoholic solvent, for example an ether. A preferred solvent is tetrahydrofuran.

According to one feature of the invention, the aryl disulfide of the general formula III in step (a) can be added to the reaction mixture at the start. The aryl disulfide used can, for example, be diphenyl disulfide.

It is an advantage of the invention that the insertion of the thioether group in step (a) of the method according to the invention takes place in good to excellent yield.

According to one feature of the invention, the thioether intermediate in step (b) can be separated off from the remains of the starting material of the general formula II and from excess reagent of the formula III or from aromatic thiol by means of start-point filtration over silica gel.

In step (c) of the method according to the invention, the sulfur of the thioether intermediate is oxidized with a reagent suitable for the oxidation of thioethers. According to one feature of the invention, the oxidation can be carried out with an alkali metal metaperiodate or an alkali metal permanganate.

The 1,2 double bond can then be introduced according to the invention through simple heating in an inert solvent. The inert solvent used in step (d) can be a substituted or unsubstituted inert aromatic solvent. A preferred solvent is xylene.

According to one feature of the invention, isolation with the end product in step (e) can take place upon cooling though crystallization in crystalline, largely pure form, from the reaction mixture.

A preferred solvent for use in step (d) of the method according to the invention is xylene. On account of the high boiling point of xylene, the reaction is rapid, and on account of the poorer solubility of azasteroids in xylene compared with toluene, spontaneous crystallization starts more easily upon cooling. As a result, work-up of the reaction mixture, particularly on the industrial scale, is greatly simplified. Finally, the end product is obtained preferably by crystallization in crystalline, largely pure form from the reaction mixture.

The resulting end product can, if necessary, be recrystallized in a further step in order to remove residual amounts of slight impurities. Solvents suitable for the recrystallization are, for example, those chosen from the group consisting of xylene, dioxane, isopropanol or a mixture of these solvents.

According to one feature of the invention, the starting material used is dihydrofinasteride (general formula II: R¹ =NR^{2′}R^{3′}, where R^{2′} is H and R^{3′} is tert-butyl, R² =H, in which - - - marks single bonds and the A and B ring of the steroid are trans linked), which reacts to give the product finasteride (general formula I: R¹ =NR^{2′}R^{3′}, where R^{2′} is H and R^{3′} is tert-butyl, R² =H and - - - marks single bonds and the A and B ring of the steroid are trans linked).

According to one feature of the invention, the alkoxides in step (a) can be used in a defined molecular ratio to the starting material, where at least one 1 mol equivalent and at most 3 mol equivalents, but preferably 2 mol equivalents, of alkoxide are used.

According to one feature of the invention, the reaction can be carried out in an ethereal solvent, preferably tetrahydrofuran, and proceed at elevated temperature, preferably the boiling temperature of the solvent.

According to one feature of the invention, turosteride is produced by the method according to the invention.

According to one feature of the invention, the 1,2-unsaturated azasteroids produced by the method according to the invention are used for producing medicaments.

Specific embodiments of the invention are illustrated below by reference to nonlimiting examples.

### EXAMPLES

### Example 1:

### Deprotonation with Sodium Methoxide and Addition of the Disulfide

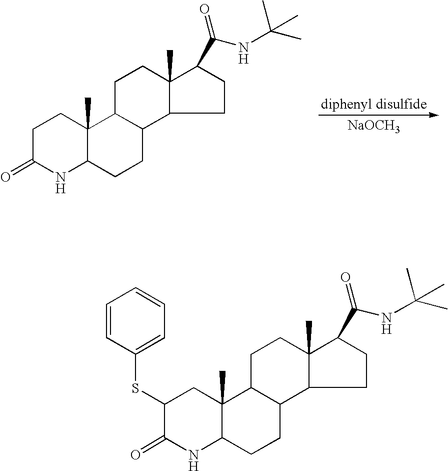 10 g of dihydrofinasteride are suspended in 100 ml of anhydrous tetrahydrofuran under an argon atmosphere (exclusion of moisture) and heated with 2.87 g of Na methoxide and 28.75 g of diphenyl disulfide at boiling for 16 h. The reaction is then cooled, the mixture is diluted with dichloroethane and 2N sulfuric acid, the phases are separated and extracted by shaking with in each case 2N sulfuric acid, 2N sodium hydroxide solution, and water, and evaporation is carried out. This gives 36.138 g of a yellowish mass which crystallizes from the melt.

### Example 2:

### Deprotonation with Sodium Methoxide and Addition of the Disulfide

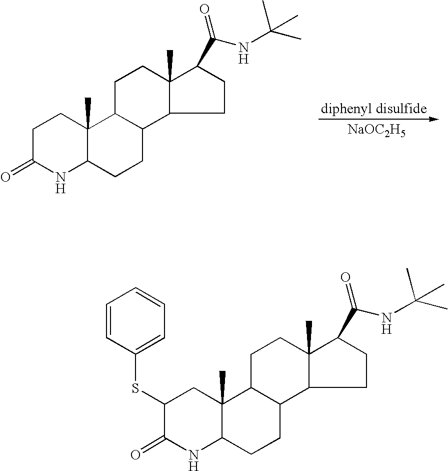 1.5 g of dihydrofinasteride are suspended in 15 ml of anhydrous tetrahydrofuran under an argon atmosphere (exclusion of moisture) and heated with 0.544 g of Na ethoxide and 4.31 g of diphenyl disulfide at boiling for 16 h.

The reaction is then cooled, the mixture is diluted with 15 ml of dichloroethane and 15 ml of 2N sulfuric acid, the phases are separated and extracted by shaking with in each case 15 ml of 2N sulfuric acid, 2N sodium hydroxide solution, and water, and evaporation is carried out. This gives 1.80 g of a yellowish mass which crystallizes from the melt.

### Example 3:

### Deprotonation with Potassium Tert-Butoxide and Addition of the Disulfide

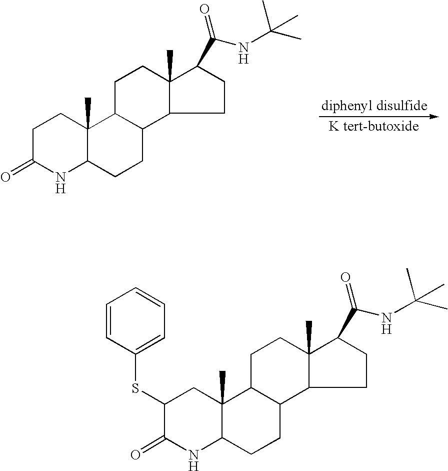 1.5 g of dihydrofinasteride are suspended in 15 ml of anhydrous tetrahydrofuran under an argon atmosphere (exclusion of moisture) and heated with 0.90 g of K tert-butoxide and 4.31 g (20.0 mmol) of diphenyl disulfide at boiling for 16 h.

The reaction is then cooled, the mixture is diluted with 15 ml of dichloroethane and 15 ml of 2N sulfuric acid, the phases are separated and extracted by shaking with in each case 15 ml of 2N sulfuric acid, 2N sodium hydroxide solution, and water, and evaporation is carried out. This gives 1.76 g of a yellowish mass which crystallizes from the melt.

### Example 4:

### Start-Point Filtration

36 g of crude mass of the thioether intermediate (Examples 1 to 3) are dissolved in 100 ml of dichloromethane, and subjected to start-point filtration over a bed of 200 g of silica gel with petroleum ether:methyl tert-butyl ether =20:1. After the elution, 12.3 g of the largely pure thioether intermediate is obtained.

### Example 5:

### Oxidation of the Thioether and Elimination

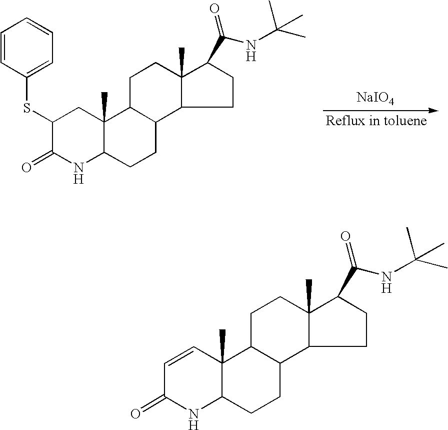

12.27 g of thioether intermediate are taken off in 130 ml of methanol with 20% water content and stirred with 13.5 g of sodium metaperiodate at room temperature for 16 h.

Then, the mixture is diluted with dichloroethane and water, the phases are separated and extracted again by shaking with dichloroethane. Following evaporation, 14.2 g of a delicate-yellow solid are obtained.

This solid is taken up in xylene and heated at reflux for 2 h. Upon cooling with stirring, crystallization starts. The crystals are filtered off with suction and then washed with xylene. This gives 8.4 g of finasteride.

### Example 6:

### Recrystallization

8 g of finasteride from Example 5 are dissolved in isopropanol with heating, inoculated upon cooling and left for 16 hours at 5° C. The crystals are filtered off with suction and dried under reduced pressure. This gives 5.6 g of finasteride.

1-17. (canceled)

18. A method of producing 1,2-unsaturated azasteroids of the general formula I, 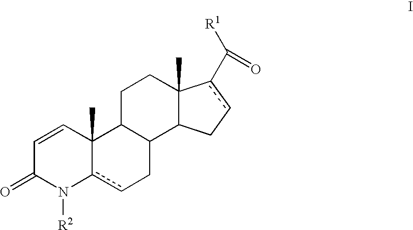
in which
R¹ is NR^{2′}R^{3′} (where R^{2′} and R^{3′}, which may be identical or different, may be H, branched or unbranched, saturated or unsaturated, substituted or unsubstituted lower alkyl having 1 to 7 carbon atoms, preferably tert-butyl, isopropyl, ethynyl, arylalkyl, benzyl, and (unsubstituted or substituted by branched or unbranched lower alkyl having 1 to 7 carbon atoms amino)carbonyl, preferably (isopropylamino)carbonyl), OR^{4′} (where R^{4′} is H, branched or unbranched, substituted or unsubstituted lower alkyl having 1 to 7 carbon atoms, preferably methyl), or nitrile;
R² is H;
and the bonds marked by - - - are single and double bonds in any combination, said method comprising the following steps:
(a) reaction of a 1,2-saturated azasteroid of the general formula II, 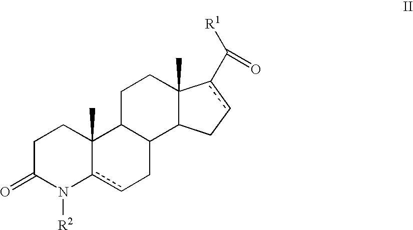
in which
R¹ is NR^{2′}R^{3′} (where R^{2′} and R^{3′}, which may be identical or different, may be H, branched or unbranched, saturated or unsaturated, substituted or unsubstituted lower alkyl having 1 to 7 carbon atoms, preferably tert-butyl, isopropyl, ethynyl, arylalkyl, benzyl, and (unsubstituted or substituted by branched or unbranched lower alkyl having 1 to 7 carbon atoms amino)carbonyl, preferably (isopropylamino)carbonyl), OR^{4′} (where R^{4′} is H, branched or unbranched, substituted or unsubstituted lower alkyl having 1 to 7 carbon atoms, preferably methyl), nitrile, halogen or pyridiniummethyl,
R² is H;
and the bonds marked by - - - are single and double bonds in any combination, as starting material with an alkali metal salt of a lower branched or unbranched alcohol in an inert solvent in the presence of an aryl disulfide of the general formula III,
Ar—S—S—Ar III
in which Ar is an unsubstituted or substituted aromatic ring;
(b) separating a resulting thioether intermediate from remains of the starting material of the general formula II and from excess reagent of the formula III or from aromatic thiol;
(c) oxidation of the sulfur of the thioether intermediate;
(d) heating the resulting product in an inert solvent;
(e) isolation of an end product from the reaction mixture; and
(f) optionally, recrystallization of the end product obtained in this way.

19. The method according to claim 18, wherein said alkali metal salt of a lower branched or unbranched alcohol used in step (a) is a sodium or potassium alkoxide.

20. The method according to claim 18, wherein said alkali metal salt of a lower branched or unbranched alcohol is sodium methoxide, sodium ethoxide or potassium tertbutoxide.

21. The method according to claim 18, wherein said inert solvent of step (a) is an ether.

22. The method according to claim 18, wherein said inert solvent of step (a) is tetrahydrofuran.

23. The method according to claim 18, wherein said aryl disulfide of the general formula III is added to the reaction mixture at the start.

24. The method according to claim 18, wherein said aryl disulfide is diphenyl disulfide.

25. The method according to claim 18, wherein said thioether intermediate is separated from remains of said starting material of the general formula II and from excess reagent of the formula III or from aromatic thiol by means of start-point filtration via silica gel.

26. The method according to claim 18, wherein sulfur of said thioether intermediate is further oxidized with an alkali metal metaperiodate or an alkali metal permanganate.

27. The method according to claim 18, wherein said inert solvent used in step (d) is a substituted or unsubstituted inert aromatic solvent.

28. The method according to claim 18, wherein said inert solvent used in step (d) is xylene.

29. The method according to claim 18, wherein said isolation of said end product in step (e) takes place upon cooling by crystallizing out in substantially pure crystalline from said reaction.

30. The method according to claim 18, wherein an elimination reaction takes place after oxidation in xylene and further wherein isolation of an end product takes place upon cooling after crystallization from the reaction solution.

31. The method according to claim 18, wherein an end product is recrystallized in step (f) from xylene, dioxane, isopropanol or any mixture of at least two of these solvents.

32. The method according to claim 18, wherein said starting material used is dihydrofinasteride (general formula II: R^{1=NR}^{2′}R^{3′}, where R^{2′} is H and R^{3′} is tert-butyl, R² is H, in which - - - marks single bonds and the A and B ring of the steroid are trans linked), which reacts to give the product finasteride (general formula I: R¹=NR^{2′}R^{3′}, where R^{2′} is H and R^{3′} is tert-butyl, R² is H, in which - - - marks single bonds and the A and B ring of the steroid are trans linked).

33. The method according to claim 18, wherein said alkoxides are used in a defined molecular ratio to the starting material, wherein at least 1 mol equivalent and at most 3 mol equivalents of alkoxide are used.

34. The method according to claim 33, wherein said alkoxides used in a defined molecular ratio to the starting material equals 2 mol equivalents.

35. The method according to claim 18, wherein said reaction is carried out in an ethereal solvent and at elevated temperatures.

36. The method according to claim 18, wherein said reaction is carried out in an ethereal solvent and at the boiling temperature of the solvent.

37. The method according to claim 35, wherein said ethereal solvent is totrahydrofuran.

38. The method according to claim 36, wherein said ethereal solvent is tetrahydrofuran

39. The method according to claim 18, wherein said end product is recrystallized from isopropanol.

40. The method according to claim 18, wherein turosteride is produced as said end product.

41. A method of producing medicaments, producing the 1,2-unsaturated azasteroids with the method according to claim 18, and using the 1,2-unsaturated azasteroids for producing the medicaments.
